(19) United States
(12) Patent Application Publication
(10) Pub. No.: US 2025/0018117 A1
(43) Pub. Date: Jan. 16, 2025

(54) Sub-assembly for a medicament delivery device

(71) Applicant: SHL Medical AG, Zug (CH)

(72) Inventors: Meng-Jhen Chiou, Taoyuan City (TW); Oscar Alexandersson, Nacka Strand (SE); Daniel Carlsson, Nacka Strand (SE); Johan Zander, Nacka Strand (SE)

(21) Appl. No.: 18/711,932

(22) PCT Filed: Nov. 17, 2022
(86) PCT No.: PCT/EP2022/082186

§ 371 Date: May 21, 2024

(30) Foreign Application Priority Data
Dec. 1, 2021 (EP) 21211646.1

(51) Int. Cl. A61M 5/20

(52) U.S. Cl. CPC A61M 5/20; A61M 2207/10

(57) ABSTRACT

The present disclosure provides a sub-assembly for a medicament delivery device configured to expel medicament from a medicament container. The sub-assembly comprises a housing and a medicament container carrier. The medicament container carrier comprises a carrier wall having an open proximal end, at least one slot, and at least a first leg portion and a second leg portion radially separated by the slot. The sub-assembly further comprises a first retaining structure of the housing, and a second retaining structure of the first and second leg portions. The first retaining structure exerts a force on the second retaining structure at least in the circumferential direction to prevent the first and second leg portions from moving radially outwards.

### CROSS-REFERENCE TO RELATED APPLICATIONS

The present application is a U.S. National Phase application pursuant to 35 U.S.C. § 371 of International Application No. PCT/EP2022/082186 filed Nov. 17, 2022, which claims priority to European Patent Application No. 21211646.1 filed Dec. 1, 2021. The entire disclosure contents of these applications are herewith incorporated by reference into the present application.

### TECHNICAL FIELD

The present disclosure generally relates to a medicament delivery device for expelling medicament from a medicament container. In particular, the present disclosure relates to a sub-assembly for a medicament delivery device, and to a method for assembling a medicament container into such sub-assembly.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various limitations do still exist in the current technology. Not least amongst these are the movement of the medicament container within the housing. In considering this, the applicant has appreciated that various developments could be made to help improve the medicament delivery devices on the market today, which are set out in more detail below.

### SUMMARY

An object of the present disclosure is thus to provide a sub-assembly for a medicament delivery device, and a medicament delivery device, which solves, or at least mitigates, problems of the prior art.

There is hence according to a first aspect of the present disclosure provided a sub-assembly for a medicament delivery device configured to expel medicament from a medicament container, the sub-assembly comprising: a housing having a proximal end and a distal end; and a medicament container carrier extending along a longitudinal axis from a proximal end to a distal end. The medicament container carrier comprises: a carrier wall having an open proximal end, at least one slot extending from the open proximal end in a distal direction, and at least a first leg portion and a second leg portion radially separated by the slot at least at the open proximal end such that the first and second leg portions are radially movable relative each other. The sub-assembly further comprises a first retaining structure extending from an inner surface of the housing, and a second retaining structure extending from an outer surface of each one of the first and second leg portions at the open proximal end of the carrier wall, wherein the first retaining structure exerts a force on the second retaining structure at least in the circumferential direction to prevent the first and second leg portions from moving radially outwards.

An effect obtainable thereby is an improved radial retainment of the first and second leg portions in the housing. Hereby, a medicament container housed in the medicament container carrier will be prevented from undesired dislocation, e.g. by being prevented from at least partly slipping out proximally of the medicament container carrier. That is, in an assembled state of the medicament delivery device in which the medicament container carrier is arranged in a final proximal position within the housing, the first retaining structure of the housing exerts a force on the second retaining structure of the medicament container carrier at least in the circumferential direction to prevent the first and second leg portions from moving radially outwards. Thus, the first and second retaining structures retain, or keep, the first and second leg portion in radial position to secure a medicament container within the medicament container carrier.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to one embodiment, the housing is tubular or substantially tubular. Thus, the inner surface of the housing is typically an inner tubular or inner cylindrical surface, or may be referred to as an inner enveloping cylindrically shaped surface.

According to one embodiment, the medicament container carrier is tubular or substantially tubular. Thus, the carrier wall of the medicament container carrier is typically cylindrically shaped. The medicament container carrier is configured to fit inside the housing.

Thus, the housing and/or the medicament container carrier may be defined by cylindrical coordinates in a cylindrical coordinate system. Thus, a radial cross section of the housing and/or the medicament container carrier may be defined by polar coordinates in a polar coordinate system. A cylindrical structure advantageously provides for rotational symmetry.

According to one embodiment, the first and second retaining structures that are arranged to prevent the first and second leg portions from moving radially outwards, are additionally arranged to prevent the first and second leg portions from moving in the circumferential direction. Thus, the first and second leg portions are locked in position, both radially and circumferentially, by the first and second retaining structures by obstructing an outwardly movement of the first and second leg portions. Thus, an outwardly radially and circumferentially movement of the first and second leg portions is obstructed by the first retaining structure and its interaction with the second retaining structure. Hereby, the first and second retaining structures obstruct undesired movement of the medicament container.

It should be understood that when referring to that the first retaining structure exerts a force on the second retaining structure at least in the circumferential direction, the exerted force comprises a force component in the circumferential direction, and may thus be referred to as a circumferential force.

According to one embodiment, the first retaining structure comprises first and second housing contact surfaces arranged at different radial positions within the inner surface of the housing, and the second retaining structure comprises first and second carrier contact surfaces arranged on the first leg portion and the second leg portion, respectively, such that the first retaining structure exerts a force at least in the circumferential direction on the second retaining structure by exerting a first force from the first housing contact surface to the first carrier contact surface, and by exerting a second force at least in the circumferential direction from the second housing contact surface to the second carrier contact surface, the second force being at least partly opposite to the first force. Hereby, the at least partly opposite forces applied to the second retaining structure by the first retaining structure efficiently obstructs the first and second leg portions from moving radially outwards. Thus, it should be understood that according to at least some embodiments, the first retaining structure comprises structure portions or surfaces radially separated from each other. Correspondingly, the second retaining structure may comprise structure portions or surfaces radially separated from each other, typically by being comprised in the first and second leg portions.

According to one embodiment, the first retaining structure extends in the radial direction from the inner surface of the housing such that each one of the first and second housing contact surfaces has a normal deviating from the radial direction. That is, the first retaining structure extends in radially inwards from the inner surface of the housing. Stated differently, each one of the first and second housing contact surfaces of the first retaining structure has a normal angled relative the radial direction. For example, as expressed in mathematical terms, a normal component of a vector extending perpendicularly from the first or second housing contact surfaces deviates from the radial direction. Such normal component of the vector typically extends at least partly in the circumferential direction.

According to one embodiment, the second retaining structure extends in the radial direction from the outer surface of each one of the first and second leg portions such that each one of the first and second carrier contact surfaces has normal deviating from the radial direction. That is, the second retaining structure extends in radially outwards from the respective outer surface of the first and second leg portions. The outer surfaces of the first and second leg portions may commonly be referred to as the outer surface of the carrier wall. Stated differently, each one of the first and second carrier contact surfaces of the second retaining structure has a normal angled relative the radial direction. For example, as expressed in mathematical terms, a normal component of a vector extending perpendicularly from the first or second carrier contact surfaces deviates from the radial direction. Such normal component of the vector typically extends at least partly in the circumferential direction.

According to one embodiment, the first housing contact surface match the first carrier contact surface, and the second housing contact surface match the second carrier contact surface.

According to one embodiment, the first retaining structure comprises at least two axially and radially extending protrusions, and the second retaining structure comprises at least two axially and radially extending protrusions, or recesses, configured to engage with the at least two protrusions of the first retaining structure. Thus, the two axially and radially extending protrusions of the first retaining structure comprises the first and second housing contact surfaces. That is, a first protrusion of the first retaining structure comprises the first housing contact surface and a second protrusion of the first retaining structure comprises the second housing contact surface. Correspondingly, the two axially and radially extending protrusions or recesses of the second retaining structure comprises the first and second carrier contact surfaces. That is, a first protrusion, or a first recess of the second retaining structure comprises the first carrier contact surface and a second protrusion, or a second recess of the second retaining structure comprises the second carrier contact surface. That is, the protrusions of the first retaining structure may be arranged to interact with corresponding protrusions of the second retaining structure, or may be arranged to interact with associated recesses of the second retaining structure.

Typically, each one of the protrusions of the first retaining structure extends radially inwards and has at least one planar lateral side, wherein the first and second housing contact surfaces are comprised in such lateral sides. The protrusions of the first retaining structure may be ribs. Correspondingly, each one of the protrusions of the second retaining structure extends radially outwards and has at least one planar lateral side, wherein the first and second carrier contact surfaces are comprised in such lateral sides. The protrusions of the second retaining structure may be ribs. Alternatively, each one of the recesses of the second retaining structure extends radially inwards and has at least one planar lateral side, wherein the first and second carrier contact surfaces are comprised in such lateral sides.

According to one alternative embodiment, the first retaining structure comprises at least two axially and radially extending recesses, and the second retaining structure comprises at least two axially and radially extending protrusions configured to engage with the at least two recesses of the first retaining structure.

According to one embodiment, the carrier wall comprises a distal end portion with an open distal end, wherein the at least one slot extends from the open proximal end to the distal end portion, the slot ending in the distal end portion prior the open distal end. Thus, the first and second leg portions are, prior to assembly of the medicament container carrier into the housing, able to flex outwardly to facilitate insertion of the medicament container into the medicament container carrier. The at least one slot typically extends along a majority of the longitudinal extension of the medicament container carrier. That is, the at least one slot has its opening in the open proximal end, extends in the distal direction, and ends in the distal end portion prior to the open distal end. For example, the distal end portion extends up to 50%, such as e.g. up to 25% of the longitudinal extension of the medicament container carrier. The cross sectional shape (radial cross section) of the carrier wall at the open distal end is circular or annular. Stated differently, the radial cross section of the carrier wall at the open distal end is a circular tube section.

According to one embodiment, the slot is a single slot. Hereby, the cross sectional shape (radial cross section) of the carrier wall at the open proximal end is C-shaped.

According to one embodiment, the open distal end comprises a distal supporting collar arranged around an outer periphery of the carrier wall to support a flange of the medicament container when the medicament container is arranged in a final proximal position within the housing. Typically, during assembly, the medicament container is inserted into the medicament container carrier to provide a medicament container sub-assembly in which a flange of the medicament container is supported by the distal supporting collar. The medicament container sub-assembly may then be inserted into the housing from the distal end until a final proximal position of the medicament container carrier is reached. The distal supporting collar thus contributes to holding the medicament container in position within the medicament container carrier.

According to one embodiment, the carrier wall comprises a proximal supporting collar arranged around an inner periphery of the carrier wall to support a neck of the medicament container when the medicament container is arranged in a final proximal position within the housing. Typically, during assembly, the medicament container is inserted into the medicament container carrier such that a shield, or an RNS (rigid needle shield), of the medicament container passes the proximal supporting collar. In more detail, the shield is able to pass the proximal supporting collar as the first and second leg portions of the proximal open end of the carrier wall is moved from a resting position to a flexed position by that the first and second leg portions are moved or flexed outwardly by the shield upon a proximally applied force from the medicament container during its insertion into the medicament container carrier. After the shield has passed the proximal supporting collar, the first and second leg portions are moved, or flexed, inwardly back to its resting position to enable the proximal supporting collar to support a neck of the medicament container. The medicament container sub-assembly may then be inserted into the housing from the distal end until a final proximal position of the medicament container carrier is reached. In the final proximal position of the medicament container carrier, the first retaining structure interacts with the second retaining structure to prevent the first and second leg portions from moving radially outwards as previously described.

According to one embodiment, the at least one slot of the medicament container carrier is a first slot, and the medicament container carrier comprises a second slot extending from the open proximal end in a distal direction opposite to the first slot, and wherein the first and second leg portions are radially separated by the first slot and the second slot at least at the open proximal end. Hereby, the ability of the first and second leg portions to flex outwardly to facilitate insertion of the medicament container into the medicament container carrier may be further improved. The second slot may be sized and dimensioned correspondingly to the first slot previously described. According to one embodiment, the cross sectional shape (radial cross section) of the carrier wall at the open proximal end is comprised of two U-shaped, or arc-shaped, sections.

According to one embodiment, the first retaining structure further comprises third and fourth housing contact surfaces arranged at different radial positions within the inner surface of the housing, such that the first, second, third and fourth housing contact surfaces are evenly distributed around a periphery of the inner surface of the housing, wherein the second retaining structure further comprises third and fourth carrier contact surfaces arranged on the first leg portion and the second leg portion, respectively, such that the first retaining structure exerts a force on the second retaining structure by further exerting a third force at least in the circumferential direction from the third housing contact surface to the third carrier contact surface, and by exerting a fourth force at least in the circumferential direction from the fourth housing contact surface to the fourth carrier contact surface, wherein the third force is at least partly opposite the fourth force. Hereby, the obstruction of the first and second leg portions from moving radially outwards owing to the at least partly opposite forces applied to the second retaining structure by the first retaining structure is further improved.

The third and fourth housing contact surfaces may be arranged in a corresponding manner as the previously described first and second housing contact surfaces. Thus, each one of the third and fourth housing contact surfaces typically has a normal deviating from the radial direction.

In other words, the first retaining structure may comprise at least two, such as four, separate first retaining structure portions, wherein each first retaining structure portion comprises one of the housing contact surfaces. Correspondingly, the second retaining structure may comprise at least two, such as four, separate second retaining structure portions, wherein each second retaining structure portion comprises one of the carrier contact surfaces. The second retaining structure portions are typically evenly distributed among the first and second leg portions.

According to one embodiment, the first retaining structure further exerts a radial force on the second retaining structure to radially press the first and second leg portions to inwards. Thus, in addition to the circumferentially applied force(s), the first retaining structure may exert a radial force to the second retaining structure. Such radially applied force may e.g. be transferred via radial contact surfaces of the inner surface of the housing and outer surface of the carrier wall.

There is according to a second aspect of the present disclosure provided a method for assembling a medicament container into a sub-assembly according to the first aspect of the present disclosure. The medicament container comprises a delivery member for expelling a medicament and a shield covering the delivery member, wherein the method comprises: inserting the medicament container in the medicament container carrier to provide a medicament container sub-assembly; inserting the medicament container sub-assembly into the housing such that the medicament container is arranged in a final proximal position within the housing, wherein the first retaining structure of the housing exerts a force at least in the circumferential direction on the second retaining structure of the medicament container carrier to prevent the first and second leg portions from moving radially outwards.

Effects and features of the second aspect of the invention are largely analogous to those described above in connection with the first aspect of the invention. Embodiments mentioned in relation to the first aspect of the invention are largely compatible with the second aspect of the invention. Thus, for example, the carrier wall may comprise a proximal supporting collar arranged around an inner periphery of the carrier wall, wherein the step of inserting the medicament container in the medicament container carrier is performed such that the shield passes the open proximal end and a neck of the medicament container is supported by the proximal supporting collar.

There is according to a third aspect of the present disclosure provided a medicament delivery device for expelling medicament from a medicament container, the medicament delivery device comprising a sub-assembly according to the first aspect of the present disclosure.

Effects and features of the third aspect of the invention are largely analogous to those described above in connection with the first aspect of the invention. Embodiments mentioned in relation to the first aspect of the invention are largely compatible with the third aspect of the invention.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

FIG. 1 schematically shows a perspective view of an example of a medicament delivery device;

FIG. 2 shows an exploded view of a sub-assembly of the medicament delivery device in FIG. 1;

FIG. 3 is a detailed perspective view of a medicament container carrier of the sub-assembly of FIG. 2;

FIG. 4A is a perspective view of a medicament container and the medicament container carrier of the sub-assembly of FIG. 2;

FIG. 4B is a perspective view of the medicament container inserted into the medicament container carrier of FIG. 4A for providing a medicament container sub-assembly;

FIG. 4C is a sectional view of the medicament container sub-assembly of FIG. 4B;

FIG. 4D is a sectional view of the medicament container inserted into the sub-assembly of FIG. 2;

FIGS. 5A-5B are radial cross sectional views at the open proximal end of the medicament container carrier of the sub-assembly shown in FIG. 2, corresponding to the state shown in FIG. 4D;

FIGS. 6A-6B are perspective views of parts of a medicament container carrier and a housing according to an alternative example of a sub-assembly for a medicament delivery device;

FIGS. 7A-7B are perspective views of parts of yet another medicament container carrier and housing according to an alternative example of a sub-assembly for a medicament delivery device;

FIG. 8A is a perspective view of an example medicament container carrier; and

FIG. 8B is a radial cross sectional view at the open proximal end of the medicament container carrier of a sub-assembly comprising the medicament container carrier of FIG. 8A.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

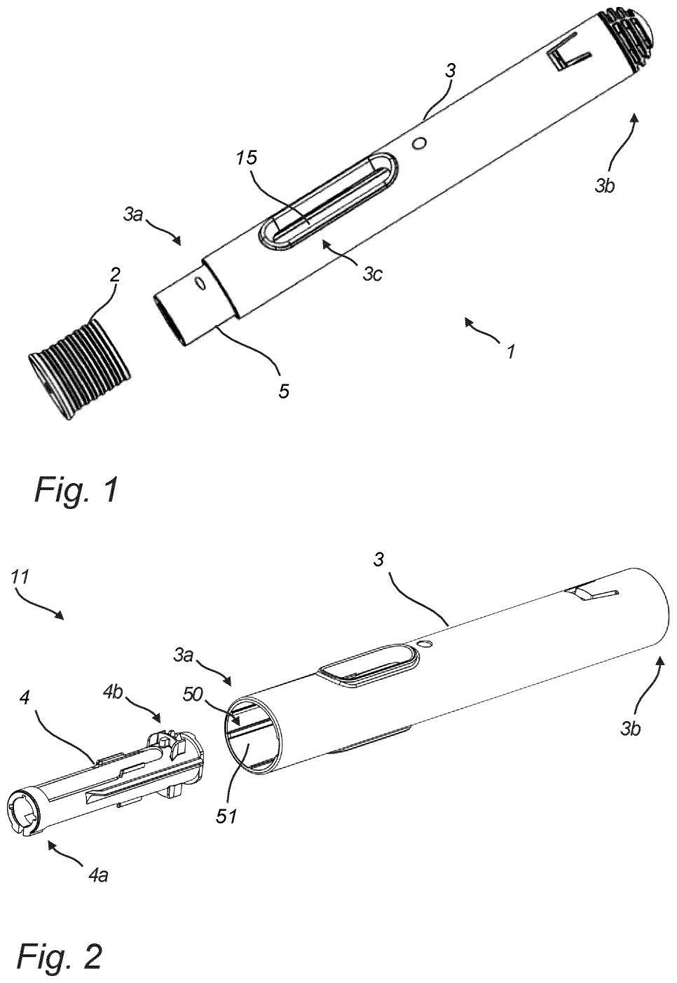

FIG. 1 shows an example of a medicament delivery device 1 configured to expel medicament from a medicament container 15 loaded in a housing 3 of the medicament delivery device 1. The housing 3 has a proximal end 3a and a distal end 3b, and a window 3c, or opening, arranged between the proximal end 3a the distal end 3b for visual inspection of the medicament container 15. The medicament delivery device 1 may e.g. be an autoinjector or pen injector. In the embodiment of FIG. 1, the medicament delivery device 1 comprises a delivery member cover 5 arranged to cover a delivery member, typically a needle, of the medicament container 15 prior to usage of the medicament delivery device 1. The delivery member cover 5 is arranged in the housing 3 and extends proximally from the proximal end 3a of the housing 3. The medicament delivery device 1 may e.g. be triggered to discharge medicament from the medicament container 15 by movement of the delivery member cover 5 further into the housing 3 resulting in an exposure of the delivery member. The medicament delivery device 1 may further comprise a cap 2 removably arranged to the delivery member cover 5.

FIG. 2 shows an exploded view of a sub-assembly 11 of the medicament delivery device 1 of FIG. 1. The sub-assembly 11 comprises the housing 3 with its proximal end 3a and distal end 3b as described with reference to FIG. 1, and a medicament container carrier 4 extending along a longitudinal axis from a proximal end 4a to a distal end 4b. The medicament container carrier 4 may, after assembly, be fixedly attached to the housing 3 of the medicament delivery device 1, e.g. by a snap lock or other conventional engagement means. The medicament container carrier 4 and the housing 3 are preferably made of the same material, e.g. plastic. The medicament container carrier 4 is configured to house and support the medicament container 15 (shown in FIG. 1).

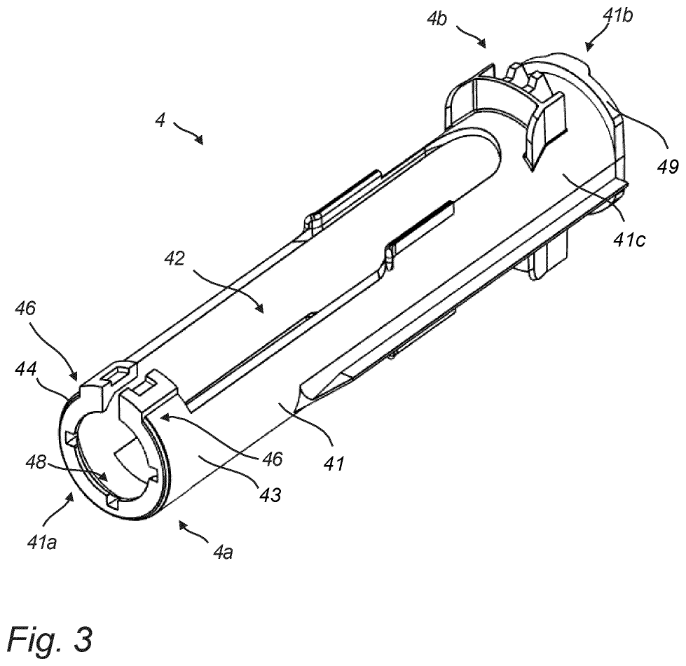

The medicament container carrier 4 is shown in greater detail in FIG. 3. The medicament container carrier 4 comprises a carrier wall 41 having an open proximal end 41a, and an open distal end 41b. The open proximal end 41a is arranged at the proximal end 4a of the medicament container carrier 4, and the open distal end 41b is arranged in a distal end portion 41c of the carrier wall 41, at the distal end 4b of the medicament container carrier 4.

The medicament container carrier 4 comprises a slot 42 extending from the open proximal end 41a in a distal direction to the distal end portion 41c. The slot 42 ends in the distal end portion 41c prior the open distal end 41b. As a result of the slot 42, the carrier wall 41 is divided into a first leg portion 43 and a second leg portion 44 radially separated by the slot 42 at the open proximal end 41a. Thus, the first and second leg portions 43, 44 are radially movable relative each other.

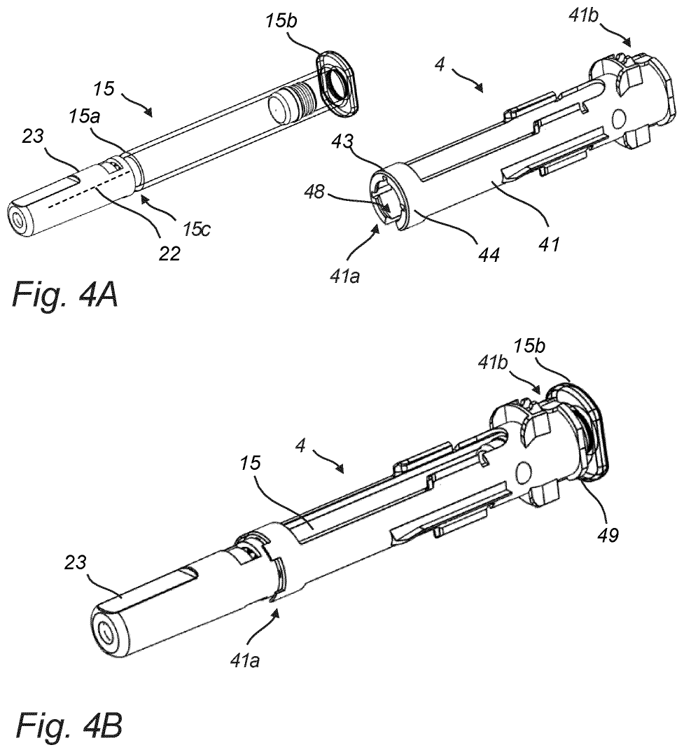

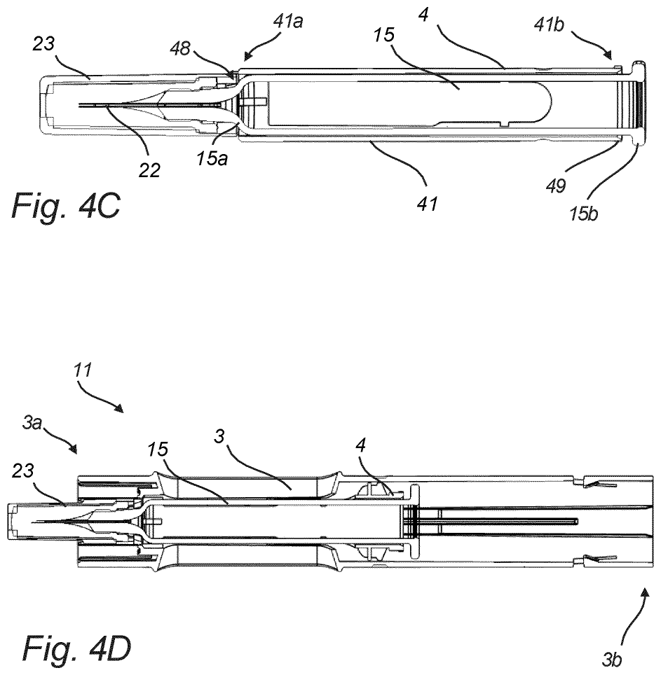

The medicament container carrier 4 comprises a distal supporting collar 49 at the open distal end 41b. The distal supporting collar 49 is arranged around an outer periphery of the carrier wall 41. The distal supporting collar 49 is configured to support a flange of the medicament container 15 as will be described with reference to FIGS. 4A-4D.

The carrier wall 41 comprises a proximal supporting collar 48 arranged around an inner periphery of the carrier wall 41 at the open proximal end 41a. The proximal supporting collar 48 is configured to support a neck of the medicament container 15 as will be described with reference to FIGS. 4A-4D.

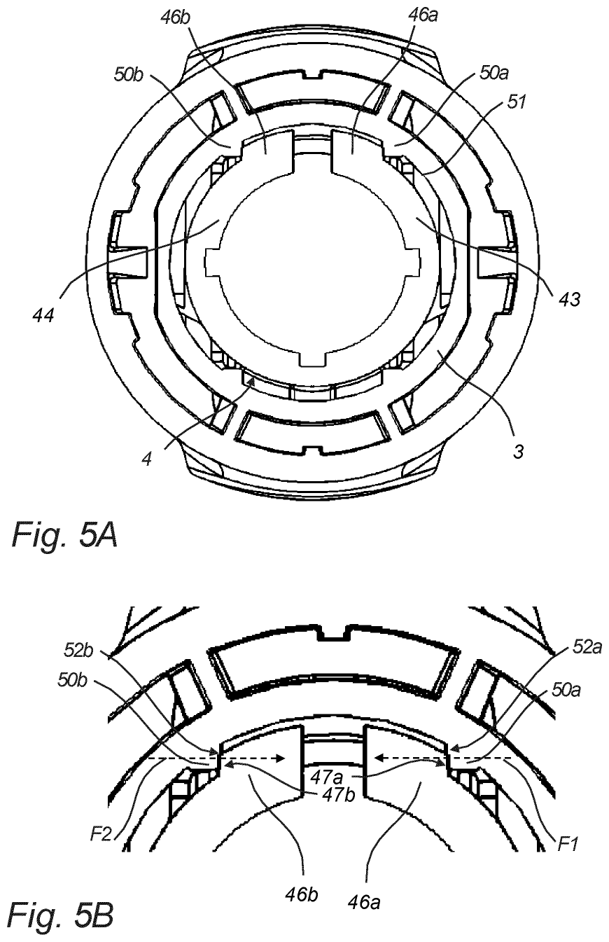

As shown in FIGS. 2 and 3, the sub-assembly 11 comprises a first retaining structure 50 extending from an inner surface 51 of the housing 3, and a second retaining structure 46 extending from an outer surface of each one of the first and second leg portions 43, 44 at the open proximal end 41a of the carrier wall 41. The first retaining structure 50 is configured to exert a force on the second retaining structure 46 at least in the circumferential direction to prevent the first and second leg portions 43, 44 from moving radially outwards, as will be further described with reference to FIGS. 5A-5B.

Turning briefly to FIGS. 4A-4D showing components of the sub-assembly 11 of FIG. 2 in various states together with a medicament container 15. In FIG. 4A, the medicament container 15 is shown in its unassembled state relative to the medicament container carrier 4.

In FIG. 4A, the medicament container 15 is shown together with a shield 23, e.g. a rigid needle shield, RNS, protecting the delivery member 22 (schematically shown as a dashed line in FIG. 4A) of the medicament container 15. The shield 23 is removably arranged relative to the body of the medicament container 15, and is arranged proximally a neck 15a of the medicament container 15. The neck 15a is separated from the shield 23 by a portion 15c of the medicament container 15 having a smaller diameter compared to the neck 15a and the shield 23. The shield 23 and the neck 15a may have the same, or substantially the same, diameter. Moreover, the medicament container 15 comprises a collar 15b arranged at the distal end of the medicament container 15.

During assembly, the medicament container 15 is inserted into medicament container carrier 4 via the open distal end 41b of the carrier wall 41. During the insertion, the shield 23 of the medicament container 15 passes the proximal supporting collar 48 via the open proximal end 41a. The shield 23 is able to pass the proximal supporting collar 48 and through the open proximal end 41a as the first and second leg portions 43, 44 are moved, or flexed, outwardly as the medicament container 15 moves proximally and the shield 23 pushes the first and second leg portions 43, 44 apart. In this respect, the diameter of the opening of the proximal open end 41a (prior to receiving the shield 23) is smaller than the diameter of the shield 23. After the shield 23 has passed the proximal supporting collar 48 and through the open proximal end 41a, the first and second leg portions are moved, or flexed, inwardly back to their initial position. The first and second leg portions 43, 44 are able to flex inwardly back to their initial position owing to the smaller diameter of the portion 15c of the medicament container 15. Hereby, the medicament container 15 is inserted into the medicament container carrier 4 to provide a medicament container sub-assembly, as is shown in FIG. 4B.

FIG. 4C shows a cross sectional view of the medicament container sub-assembly of FIG. 4B along the longitudinal direction. Here, it is clearly shown that the proximal supporting collar 48 supports the neck 15a of the medicament container 15. In FIGS. 4B-4C, the medicament container 15 is arranged in a final proximal position within the medicament container carrier 4. Moreover, the distal supporting collar 49 is configured to support the flange 15b of the distal end of the medicament container 15. As shown in FIG. 4C, there may be a small play between the flange 15b and the distal supporting collar 49.

The medicament container sub-assembly of FIGS. 4B-4C may then be inserted into the housing 3 from the distal end 3b until a final proximal position of the medicament container carrier 4 is reached within the housing 3, which is shown in FIG. 4D. In the final proximal position of the medicament container carrier 4 within the housing 3, the first retaining structure 50 interacts with the second retaining structure 46 (shown separately in FIGS. 2-3) to prevent the first and second leg portions 43, 44 from moving radially outwards.

A method for assembling the medicament container 15 with the sub-assembly 11 as shown in FIGS. 4A-4D may be described as follows. In a first step S1, the medicament container 15 is inserted into the medicament container carrier 4 to provide a medicament container sub-assembly. In a second step S2, the medicament container sub-assembly is inserted into the housing 3 such that the medicament container 15, and the medicament container carrier 4, are arranged in their respective final proximal position within the housing 3. Here, the first retaining structure 50 of the housing 3 exerts at least a force at least in the circumferential direction on the second retaining structure 46 of the medicament container carrier 4 to prevent the first and second leg portions 43, 44 from moving radially outwards. The step S1 of inserting the medicament container 15 in the medicament container carrier 4 is typically performed such that the shield 23 passes the open proximal end 41a and the neck 15a of the medicament container 15 is supported by the proximal supporting collar 48, as described with reference to FIGS. 4B-4C.

The interaction of the first retaining structure 50 with the second retaining structure 46 to prevent the first and second leg portions 43, 44 from moving radially outwards will now be described in further detail.

FIG. 5A is showing a radial cross section at the open proximal end 41a of the sub-assembly 11 in a state shown in FIG. 4D. That is, in FIG. 5A, the medicament container carrier 4 houses the medicament container 15 (not shown in FIG. 5A), and is arranged in its final proximal position within the housing 3.

As shown in FIG. 5A, the first retaining structure comprises two protrusions 50a, 50b arranged at different radial positions within the inner surface 51 of the housing 3. Each one of the two protrusions 50a, 50b of the first retaining structure extends axially as well as radially inwardly towards the medicament container carrier 4. The second retaining structure comprises two protrusion 46a, 46b arranged on the first and second leg portions 43, 44, respectively. The first protrusion 46a of the second retaining structure is extending from an outer surface of the first leg portion 43, and the second protrusion 46b of the second retaining structure is extending from an outer surface of the second leg portion 44. Each one of the two protrusion 46a, 46b of the second retaining structure extends axially as well as radially outwardly towards the housing 3. The two protrusion 46a, 46b of the second retaining structure are configured to engage with the at least two protrusions 50a, 50b of the first retaining structure.

FIG. 5B shows an enlarged view of the first and second protrusions 50a, 50b of the first retaining structure and the first and second protrusions 46a, 46b of the second retaining structure. The first protrusion 50a of the first retaining structure comprises a first housing contact surface 52a, and the second protrusion 50b of the first retaining structure comprises a second housing contact surface 52b. Correspondingly, the first protrusion 46a of the second retaining structure comprises a first carrier contact surface 47a arranged on the first leg portion 43, and the second protrusion 46b of the second retaining structure comprises a second carrier contact surface 47b arranged on the second leg portion 44. Hereby, the first retaining structure is arranged to exert forces on the second retaining structure by exerting a first force F1 from the first housing contact surface 52a to the first carrier contact surface 47a, and by exerting a second force F2 from the second housing contact surface 52b to the second carrier contact surface 47b. As each one of the first and second housing contact surfaces 52a, 52b, and each one of the first and second carrier contact surfaces 47a, 47b has a normal deviating from the radial direction, each one the resulting first and second forces F1, F2 has at least one force component in the circumferential direction. Thus, the first force F1 may be referred to as a first circumferential force, and the second force F2 may be referred to as a second circumferential force. As is obvious from FIG. 5B, as the first housing contact surface 52a is substantially parallel to the second housing contact surface 52b, and is arranged on an opposite side of the first and second carried contact surfaces 47a, 47b, the second force F2 is substantially opposite to the first force F1.

Thus, by the configuration of the first and second retaining structure as shown in FIGS. 5A-5B, an outwardly radially and circumferentially movement of the first and second leg portions 43, 44 is obstructed by the housing 3. Hereby, the first and second retaining structures may prevent undesired movement of the medicament container.

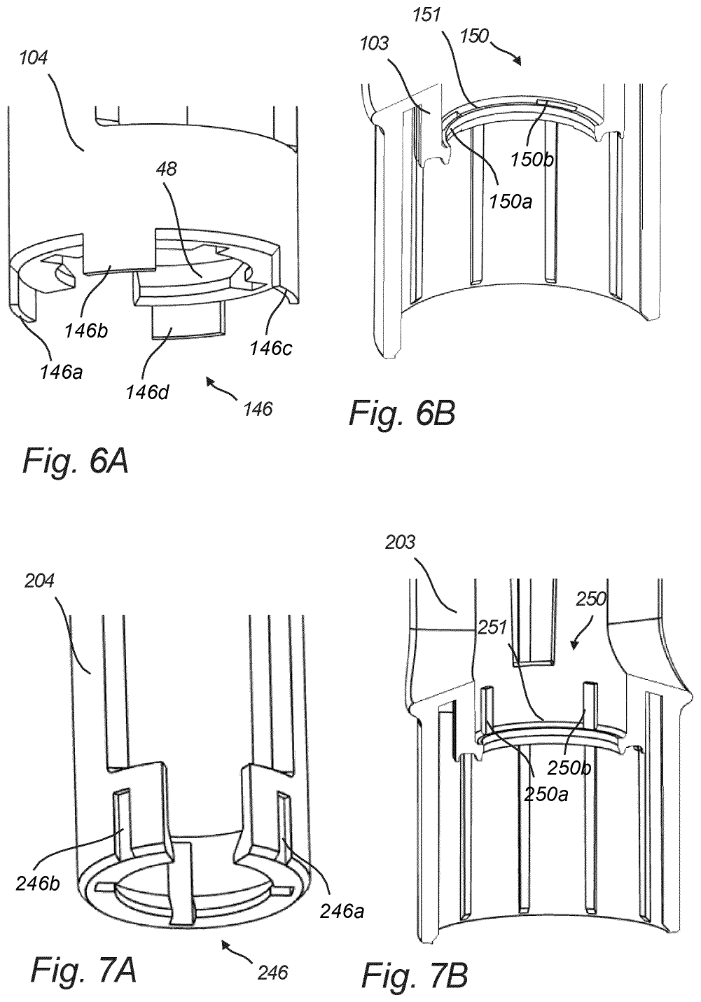

Two alternative embodiments of the sub-assembly 11 of FIGS. 5A-5B are shown in FIGS. 6A-6B and FIGS. 7A-7B. In FIG. 6A, the proximal end of a medicament container carrier 104 is shown, and in FIG. 6B the proximal end of a housing 103 is shown. The medicament container carrier 104 of FIG. 6A, and the housing 103 of FIG. 6B, are in large corresponding to the medicament container carrier 4 and the housing 3 of FIGS. 5A-5B, why only the differences are described below. Thus, the medicament container carrier 104 may form a corresponding sub-assembly together with the housing 103.

For the medicament container carrier 104 in FIG. 6A, the corresponding second retaining structure 146 are comprised of four protrusions 146a, 146b, 146c, 146d extending proximally pass the proximal supporting collar 48. The four protrusions 146a-d have an extension in the radial direction as well as in the axial direction, and are configured to interact with a corresponding first retaining structure 150 of the housing 103, shown in FIG. 6B. The first retaining structure 150 of the housing 103 are comprised of four recesses 150a, 150b (for which only two are shown in FIG. 6B) extending radially inwards in the inner surface 151 of the housing 103. Thus, the protrusions 146a, 146b, 146c, 146d of the second retaining structure 146 are configured to engage with the recesses 150a, 150b of the first retaining structure 150 in a corresponding manner as described with reference to FIGS. 5A-5B.

In FIG. 7A, the proximal end of a medicament container carrier 204 is shown, and in FIG. 7B the proximal end of a housing 203 is shown. The medicament container carrier 204 of FIG. 7A, and the housing 204 of FIG. 7B, are in large corresponding to the medicament container carrier 4 and the housing 3 of FIGS. 5A-5B, why only the differences are described below. Thus, the medicament container carrier 204 may form a corresponding sub-assembly together with the housing 203.

For the medicament container carrier 204 in FIG. 7A, the corresponding second retaining structure 246 are comprised of four recess 246a, 246b (for which only two are shown in FIG. 7A). The recesses 246a, 246b have an extension in the radial direction as well as in the axial direction and are configured to interact with a corresponding first retaining structure 250 of the housing 203, shown in FIG. 7B. The first retaining structure 250 of the housing 203 are comprised of four protrusions 250a, 250b (for which only two are shown in FIG. 7B) extending radially outwards from the inner surface 251 of the housing 203. Thus, the recesses 246a, 246b of the second retaining structure 246 are configured to engage with the protrusions 250a, 250b of the first retaining structure 250 in a corresponding manner as described with reference to FIGS. 5A-5B.

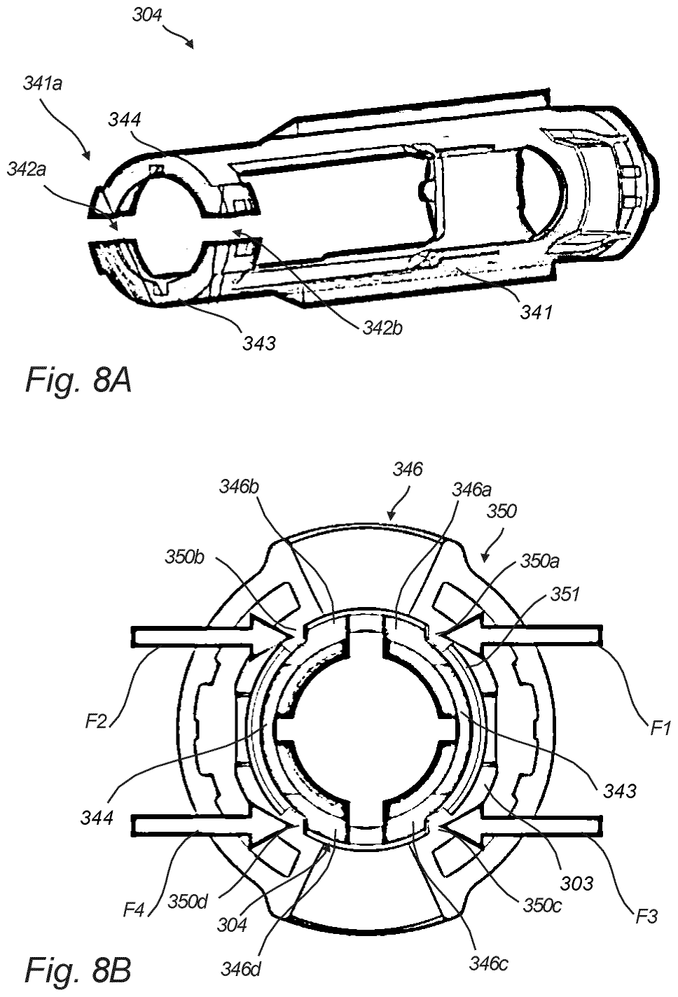

FIG. 8A shows an alternative embodiment of a medicament container carrier 304. In large, the medicament container carrier 304 corresponds to the medicament container carrier 4 of FIG. 3 why only the differences are described below. Thus, the medicament container carrier 304 may form a corresponding sub-assembly together with the housing 3 of FIG. 2, or the housing 303 of FIG. 8B. In the embodiment of FIG. 8A, the medicament container carrier 304 comprises a first slot 342a and a second slot 342b extending from the open proximal end 341a in a distal direction opposite to the first slot 342a. The corresponding first and second leg portions 343, 344 of the carrier wall 341 are thus radially separated by the first slot 342a and the second slot 342b at the open proximal end 341a. Hereby, the shield 23 to the medicament container 15 may more easily pass through the open proximal end 341a.

Corresponding to the cross section for the medicament container carrier 4 shown in FIG. 5A, the cross section of the medicament container carrier 304 is shown in FIG. 8B. Thus, the medicament container carrier 304 is arranged to interact with a corresponding housing 303 as will be described in the following.

As shown in FIG. 8B, the first retaining structure 350 comprises four protrusions 350a, 350b, 350c, 350d arranged at different radial positions within the inner surface 351 of the housing 303. Each one of the four protrusions 350a, 350b, 350c, 350d of the first retaining structure extends axially as well as radially inwardly towards the medicament container carrier 304. The second retaining structure 346 comprises four corresponding protrusion 346a, 346b, 346c, 346d arranged on the first and second leg portions 343, 344, respectively. The first and third protrusion 346a, 346c of the second retaining structure is extending from an outer surface of the first leg portion 343, and the second and fourth protrusions 346b, 346d of the second retaining structure is extending from an outer surface of the second leg portion 344. Each one of the four protrusion 346a, 346b, 346c, 346d of the second retaining structure extends axially as well as radially outwardly towards the housing 3. The four protrusion 346a, 346b, 346c, 346d of the second retaining structure are configured to engage with the four protrusions 350a, 350b, 350c, 350d of the first retaining structure in a corresponding manner as described with reference to FIGS. 5A-5B. Thus, the four protrusions 350a, 350b, 350c, 350d of the first retaining structure comprises first, second, third and fourth housing contact surfaces arranged at different radial positions within the inner surface 351 of the housing 303, such that the first, second, third and fourth housing contact surfaces are evenly distributed around a periphery of the inner surface 351 of the housing 303. Correspondingly, the four protrusion 346a, 346b, 346c, 346d of the second retaining structure comprises first, second, third and fourth carrier contact surfaces arranged on the first leg portion 343 and the second leg portion 344, respectively, such that the first retaining structure exerts forces on the second retaining structure by exerting a first force from the first housing contact surface to the first carrier contact surface, by exerting a second force from the second housing contact surface to the second carrier contact surface, by exerting a third force from the third housing contact surface to the third carrier contact surface, and by exerting a fourth force from the fourth housing contact surface to the fourth carrier contact surface. As each one of the first to fourth housing contact surfaces, and each one of the first to fourth carrier contact surfaces has a normal deviating from the radial direction, each one the resulting first to fourth forces has at least one force component in the circumferential direction. Thus, the first force may be referred to as a first circumferential force, the second force may be referred to as a second circumferential force, the third force may be referred to as a third circumferential force, and the fourth force may be referred to as a fourth circumferential force. In a corresponding manner as to FIGS. 5A-5B, the second force is substantially opposite to the first force, and the third force is substantially opposite the fourth force.

The delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims. For example, the drive member could be motor driven or manually driven instead of having an automatic spring-loaded structure.

1-15. (canceled)

16. A sub-assembly for a medicament delivery device configured to expel medicament from a medicament container, the sub-assembly comprising:
a housing having a proximal end and a distal end; and
a medicament container carrier extending along a longitudinal axis from a proximal end to a distal end, the medicament container carrier comprising a carrier wall having an open proximal end, at least one slot extending from the open proximal end in a distal direction, and at least a first leg portion and a second leg portion radially separated by the slot at least at the open proximal end such that the first and second leg portions are radially movable relative each other,
wherein a first retaining structure extends from an inner surface of the housing, and a second retaining structure) extends from an outer surface of each one of the first and second leg portions at the open proximal end of the carrier wall, wherein the first retaining structure exerts a force on the second retaining structure at least in the circumferential direction to prevent the first and second leg portions from moving radially outwards.

17. The sub-assembly according to claim 16, wherein the first retaining structure comprises first and second housing contact surfaces arranged at different radial positions within the inner surface of the housing, and wherein the second retaining structure comprises first and second carrier contact surfaces arranged on the first leg portion and the second leg portion, respectively, such that the first retaining structure exerts a force on the second retaining structure by exerting a first force at least in the circumferential direction from the first housing contact surface to the first carrier contact surface, and by exerting a second force at least in the circumferential direction from the second housing contact surface to the second carrier contact surface, the second force being at least partly opposite to the first force.

18. The sub-assembly according to claim 17, wherein the first retaining structure extends in the radial direction from the inner surface of the housing such that each one of the first and second housing contact surfaces has a normal deviating from the radial direction.

19. The sub-assembly according to claim 17, wherein the second retaining structure extends in the radial direction from the outer surface of each one of the first and second leg portions such that each one of the first and second carrier contact surfaces has normal deviating from the radial direction.

20. The sub-assembly according to claim 16, wherein the first retaining structure comprises at least two axially and radially extending protrusions, and the second retaining structure comprises at least two axially and radially extending protrusions, or recesses, configured to engage with the at least two protrusions of the first retaining structure.

21. The sub-assembly according to claim 16, wherein the first retaining structure comprises at least two axially and radially extending recesses, and the second retaining structure comprises at least two axially and radially extending protrusions configured to engage with the at least two recesses of the first retaining structure.

22. The sub-assembly according to claim 16, wherein the carrier wall comprises a distal end portion with an open distal end, and wherein the at least one slot extends from the open proximal end to the distal end portion, the slot ending in the distal end portion prior the open distal end.

23. The sub-assembly according to claim 22, wherein the open distal end comprises a distal supporting collar arranged around an outer periphery of the carrier wall to support a flange of the medicament container when the medicament container is arranged in a final proximal position within the housing.

24. The sub-assembly according to claim 16, wherein the carrier wall comprises a proximal supporting collar arranged around an inner periphery of the carrier wall to support a neck of the medicament container when the medicament container is arranged in a final proximal position within the housing.

25. The sub-assembly according to claim 16, wherein the at least one slot of the medicament container carrier is a first slot, and the medicament container carrier comprises a second slot extending from the open proximal end in a distal direction opposite to the first slot, and wherein the first and second leg portions are radially separated by the first slot and the second slot at least at the open proximal end.

26. The sub-assembly according to claim 16, wherein the first retaining structure further comprises third and fourth housing contact surfaces arranged at different radial positions within the inner surface of the housing, such that the first, second, third and fourth housing contact surfaces are evenly distributed around a periphery of the inner surface of the housing, and wherein the second retaining structure further comprises third and fourth carrier contact surfaces arranged on the first leg portion and the second leg portion, respectively, such that the first retaining structure exerts a force on the second retaining structure by further exerting a third force at least in the circumferential direction from the third housing contact surface to the third carrier contact surface, and by exerting a fourth force at least in the circumferential direction from the fourth housing contact surface to the fourth carrier contact surface, wherein the third force is at least partly opposite the fourth force.

27. The sub-assembly according to claim 16, wherein the first retaining structure further exerts a radial force on the second retaining structure to radially press the first and second leg portions inwards.

28. A method for assembling a medicament container into a sub-assembly according to claim 16, the medicament container comprising a delivery member for expelling a medicament and a shield covering the delivery member, wherein the method comprises:
inserting the medicament container in the medicament container carrier to provide a medicament container sub-assembly; and
inserting the medicament container sub-assembly into the housing such that the medicament container is arranged in a final proximal position within the housing, wherein the first retaining structure of the housing exerts a force at least in the circumferential direction on the second retaining structure of the medicament container carrier to prevent the first and second leg portions from moving radially outwards.

29. The method according to claim 28, wherein the carrier wall comprises a proximal supporting collar arranged around an inner periphery of the carrier wall, and wherein the step of inserting the medicament container in the medicament container carrier is performed such that the shield passes the open proximal end and a neck of the medicament container is supported by the proximal supporting collar.

30. A medicament delivery device for expelling medicament from a medicament container, the medicament delivery device comprising a sub-assembly according to claim 16.
